(19) United States
(12) Patent Application Publication
(10) Pub. No.: US 2012/0239017 A1
(43) Pub. Date: Sep. 20, 2012

(54) OPTICAL APPARATUS AND OPERATING METHOD THEREOF

(76) Inventors: William Wang, Taoyuan City (TW); Chung-Cheng Chou, Luzhu Township (TW)

(21) Appl. No.: 13/419,999

(22) Filed: Mar. 14, 2012

(30) Foreign Application Priority Data
Mar. 15, 2011 (TW) 100108758

(51) Int. Cl. A61B 18/20; A61N 5/067

(52) U.S. Cl. 606/9; 607/89

(57) ABSTRACT

An optical apparatus is disclosed. The optical apparatus includes an optical emitting module, a sensing module, and a processing module. The optical emitting module is used to emit a laser toward a specific region of skin surface. The sensing module is used to sense tissue distribution information under the specific region of skin surface. The processing module is used to adjust at least one laser parameter used during the optical emitting module emits the laser toward the specific region of skin surface according to the tissue distribution information.

### BACKGROUND OF THE INVENTION

1. Field of the Invention

The invention relates to optics; in particular, to an optical apparatus and operating method thereof capable of correspondingly adjusting laser parameters used in the speckle and mole removing process according to the actual speckle and mole distribution on the skin of the patient.

2. Description of the Prior Art

In recent years, with the development of optical technologies, various kinds of optical equipments are developed and applied in many regions in daily life, such as optical inspection and laser beauty.

In general, the optical laser apparatus applied to skin treatment can perform the processes of whitening, speckle and mole removing, and tattoo removing on the skin surface. However, in fact, the regions of the skin affected by speckle, mole, and tattoo pattern are not only distributed on the surface layer of the skin, they will be also distributed in the regions below the surface layer of the skin. For example, the freckle shown on the skin is distributed on the surface layer of the skin; therefore, the ruby laser can be used to remove the freckle. The tattoo is artificial coloring speckle distributed into the dermal tissue; therefore, the laser using more energy is necessary.

In current practical laser freckle removing process, lasers of different types and wavelengths will be provided for different depths and processes, however, the energy and action time of the laser used in these processes are still referenced based on the previous statistics data without referencing the actual speckle and mole distribution of the patient. Therefore, the conditions of human discomfort (excessive laser energy) or entire process delay (insufficient laser energy) will be usually caused by the errors or subjective factors of the operator, and these drawbacks should be overcome.

Therefore, the invention provides an optical apparatus and operating method thereof to solve the above-mentioned problems occurred in the prior arts.

### SUMMARY OF THE INVENTION

An embodiment of the invention is an optical apparatus. In this embodiment, the optical apparatus includes an optical emitting module, a sensing module, and a processing module. The optical emitting module is used to emit a laser toward a specific region of a skin surface. The sensing module is used to sense a tissue distribution information under the specific region of the skin surface. The processing module is used to adjust at least one laser parameter used during the optical emitting module emits the laser toward the specific region of the skin surface according to the tissue distribution information.

In practical applications, the optical emitting module emits the laser toward the specific region of the skin surface to remove the speckle, mole, or tattoo distributed on the specific region and the tissue below it. The at least one laser parameter includes a spot size, a wavelength, an emission energy, and an action time used during the optical emitting module emits the laser. In fact, the sensing module can perform a deep inspection on the specific region of the skin surface through an optical interference technology.

Another embodiment of the invention is an optical apparatus operating method. In this embodiment, the optical apparatus is used to emit a laser to treat a specific region of a skin surface. The optical apparatus operating method includes steps of (a) sensing a tissue distribution information under the specific region of the skin surface; (b) adjusting at least one laser parameter used during the optical emitting module emits the laser toward the specific region of the skin surface according to the tissue distribution information.

Compared to the prior arts, the optical apparatus and operating method thereof disclosed in the invention uses the optical interference technology (e.g., the optical coherence tomography (OCT) technology) to perform a deep inspection on the treated region of the skin surface, so that the actual speckle and mole distribution of the patient can be obtained, and the laser parameter used during the optical apparatus emits the laser can be adjusted accordingly to reach the optimal condition suitable for the patient.

Therefore, the optical apparatus and operating method thereof disclosed in the invention can effectively avoid the conditions of human discomfort (excessive laser energy) or entire process delay (insufficient laser energy) caused by the errors or subjective factors of the operator, not only the drawbacks of conventional laser speckle removing process can be effectively improved, but also the satisfaction of the consumers to the laser speckle removing process can be largely increased.

The advantage and spirit of the invention may be understood by the following detailed descriptions together with the appended drawings.

### BRIEF DESCRIPTION OF THE APPENDED DRAWINGS

FIG. 1 illustrates a function block diagram of the optical apparatus in the first embodiment of the invention.

FIG. 2 illustrates a schematic diagram of the optical apparatus performing optical sensing on the specific region of the skin surface and emitting laser to treat the specific region.

FIG. 3A and FIG. 3B illustrate a top view and a side view of selecting the specific region to be optically treated on the skin surface.

FIG. 3C and FIG. 3D illustrate a top view and a side view of grid positioning the specific region and dividing the specific region into a plurality of sub-regions.

FIG. 4 illustrates a flowchart of the optical apparatus operating method in the second embodiment of the invention.

FIG. 5 illustrates a detailed flowchart of step 10 of FIG. 4.

FIG. 6 illustrates a flowchart of the optical apparatus performing the laser speckle removing process.

### DETAILED DESCRIPTION OF THE INVENTION

The invention provides an optical apparatus and operating method thereof. The optical apparatus of the invention uses an optical interference technology (e.g., an optical coherence tomography (OCT) technology) to perform a deep inspection on the treated region of the skin surface, so that the actual speckle and mole distribution of the patient can be obtained, and the laser parameter used during the optical apparatus emits the laser can be adjusted accordingly to reach the optimal condition suitable for the patient to improve the drawbacks of the conventional speckle removing process.

A first embodiment of the invention is an optical apparatus. In this embodiment, the main function of the optical apparatus is to emit the laser most suitable for the actual speckle and mole distribution of the patient to the specific region of the skin surface, so that the speckle, mole, and tattoo distributed on the specific region and the tissue below it will be smoothly removed, and the patient will not feel uncomfortable.

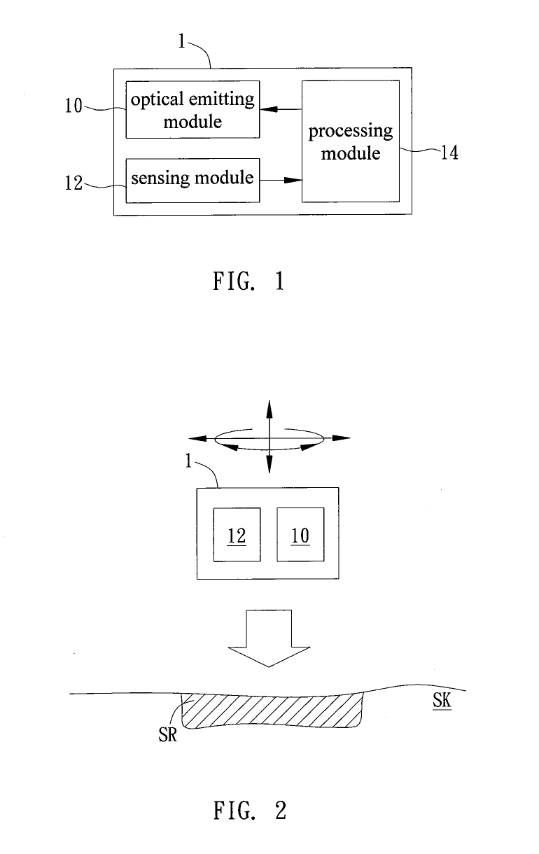

Please refer FIG. 1. FIG. 1 illustrates a function block diagram of the optical apparatus in this embodiment. As shown in FIG. 1, the optical apparatus 1 includes an optical emitting module 10, a sensing module 12, and a processing module 14. The processing module 14 is coupled to the optical emitting module 10 and the sensing module 12.

Next, the modules of the optical apparatus 1 will be introduced in detail respectively as follows.

The optical emitting module 10 is used to emit a laser toward a specific region of a skin surface. In fact, the type of the laser emitted by the optical emitting module 10 has no specific limitations, it is determined based on practical needs. It should be noticed that the laser parameters used during the optical emitting module 10 emits the laser, such as a spot size, a wavelength, an emission energy, and an action time, are controlled by the processing module 14.

The sensing module 12 is used to sense a tissue distribution information under the specific region of the skin surface. In fact, the above-mentioned tissue distribution information can include the tissue structure and the distribution of the speckle, mole, and tattoo to be removed in the surface skin layer and the dermis layer of the skin, so that the detailed information of the actual distribution of the speckle, mole, and tattoo of the patient will be provided to the processing module 14. In this embodiment, the sensing module 12 can be the contact-type sensing module capable of contacting with the surface layer of the skin, such as an optical-type sensing module, an electrode-type sensing module, or an ultrasound-type sensing module; or the sensing module 12 can be the non-contact-type sensing module, such as an optical-type sensing module, but not limited to this.

Please refer to FIG. 2. FIG. 2 illustrates a schematic diagram of the optical apparatus 1 performing optical sensing on the specific region of the skin surface and emitting laser to treat the specific region. As shown in FIG. 2, if the operator has already selected the specific region SR to be optically treated on the skin surface SK through a visual way or other ways, the optical apparatus 1 will use the sensing module 12 to perform the optical sensing on the specific region SR of the skin surface SK to obtain the tissue distribution information under the specific region SR of the skin surface SK. Then, the optical apparatus 1 will use the optical emitting module 10 to emit laser toward the specific region SR of the skin surface SK. That is to say, it is the specific region SR to be optically treated on the skin surface SK that the sensing module 12 and the optical emitting module 10 of the optical apparatus 1 both act on.

It should be noticed, as shown in FIG. 2, the optical apparatus 1 can have functions of movement and rotation, and the optical emitting module 10 can have different ways to emit laser. For example, the optical emitting module 10 can emit lasers with a fixed tilt angle; or the optical emitting module 10 can emit lasers with different angles in a rotation way, or even other ways without specific limitations. That is to say, the optical emitting module 10 or the sensing module 12 can provide independent functions of movement and rotation based on practical applications or cost consideration. In addition, when the optical emitting module 10 emits laser to the specific region SR, the sensing module 12 can be shut down or kept in ON state for observation without specific limitations.

Then, the following examples will be used to describe how the sensing module 12 performs optical sensing on the specific region SR of the skin surface SK. It should be noticed that the sensing module 12 can also performs optical sensing on the specific region SR of the skin surface SK in other ways. It is not limited to this case.

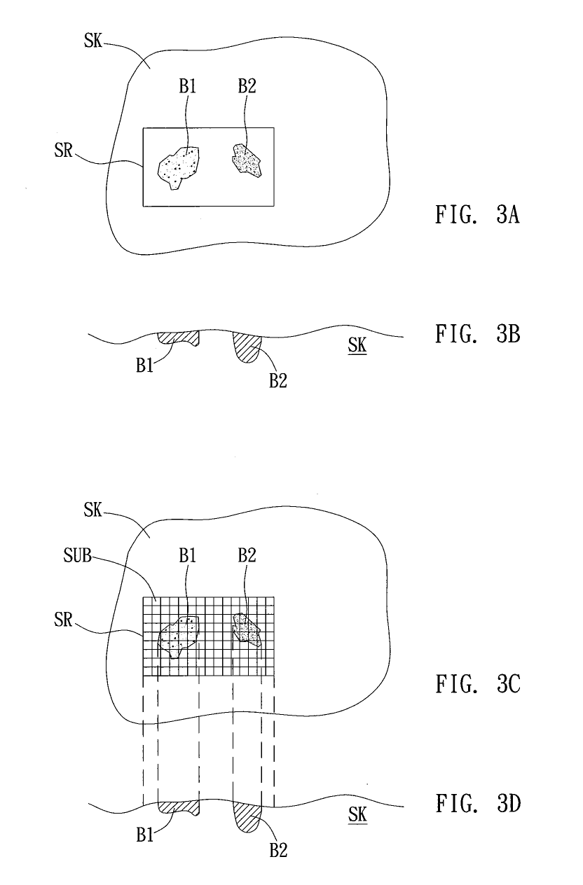

As shown in FIG. 3A and FIG. 3B, the operator selects the specific region SR to be optically treated on the skin surface SK in a visual way, and the specific region SR includes the speckles B1 and B2. Wherein, since the distribution range of the speckle B1 is larger, but the depth of the speckle B1 is smaller, the color of the speckle B1 is lighter; since the distribution range of the speckle B2 is smaller, but the depth of the speckle B2 is larger, the color of the speckle B2 is darker.

In practical applications, the sensing module 12 can directly perform a deep inspection on the specific region SR, or the sensing module 12 can firstly divide the specific region SR into a plurality of sub-regions SUB, and then perform the deep inspection on the plurality of sub-regions SUB respectively. In practical applications, the sensing module 12 can use the optical interference technology (e.g., the OCT technology) to perform the deep inspection on the plurality of sub-regions SUB. The longitudinal inspection depth of the sensing module 12 is usually 2-3 mm, and the wavelength of the light used can be 1300 nm or 840 nm, but not limited to this.

Please refer to FIG. 3C and FIG. 3D. FIG. 3C and FIG. 3D illustrate a top view and a side view of the sensing module 12 grid positioning the specific region SR and dividing the specific region SR into a plurality of sub-regions SUB. Wherein, since the distribution range of the speckle B1 is larger than the distribution range of the speckle B2, the number of sub-regions SUB covered by the speckle B1 is larger than the number of sub-regions SUB covered by the speckle B2. In fact, the sensing module 12 can use a micro-camera unit (not shown in the figures) to perform the above-mentioned grid positioning process, but not limited to this. In addition, the sensing module 12 can also used other methods to divide the specific region SR into a plurality of sub-regions SUB, not limited to the grid positioning method used in this embodiment.

After the sensing module 12 divides the specific region SR into a plurality of sub-regions SUB, the sensing module 12 will perform the deep inspection on the plurality of sub-regions SUB respectively to obtain the tissue distribution information related to each sub-region SUB. Therefore, the sensing module 12 can obtain the tissue distribution information of the sub-regions SUB covered by the speckles B1 and B2 respectively, and transmit the tissue distribution information to the processing module 14.

Similarly, in practical applications, the sensing module 12 can also use the above-mentioned methods to perform inspection to the mole or tattoo on the skin to obtain the tissue distribution information of the sub-regions SUB covered by the mole or tattoo.

After the tissue distribution information of each sub-region SUB is transmitted to the processing module 14, the processing module 14 will determine the value of the laser parameter used during the optical emitting module 10 emits lasers to each sub-region SUB of the specific region SR according to the tissue distribution information of each sub-region SUB, and the processing module 14 will also adjust the laser parameter of the optical emitting module 10 accordingly. In fact, the above-mentioned laser parameter can be a spot size, a wavelength, an emission energy, and an action time used during the optical emitting module 10 emits the laser, but not limited to this.

For example, if the processing module 14 knows that the distribution depth of a speckle located in the sub-region SUB is quite deep based on the sub-region SUB of the sub-region SUB, in order to effectively remove the speckle, the processing module 14 must increase the emission energy of the optical emitting module 10 emitting the laser to the sub-region SUB or increase the action time of the optical emitting module 10 emitting the laser to the sub-region SUB.

On the contrary, if the processing module 14 knows that the distribution depth of a speckle located in the sub-region SUB is quite swallow based on the sub-region SUB of the sub-region SUB, the processing module 14 must decrease the emission energy of the optical emitting module 10 emitting the laser to the sub-region SUB or shorten the action time of the optical emitting module 10 emitting the laser to the sub-region SUB to prevent the treated patient from discomfort or pain.

In practical applications, after the sensing module 12 finishes the grid positioning of the specific region SR, the sensing module 12 will perform the OCT on the specific region SR, and transmit the OCT result to the processing module 14, so that the processing module 14 can adjust the laser parameter of the optical emitting module 10. When the optical emitting module 10 emits laser to the specific region SR, the sensing module 12 can be shut down or kept in ON state for observation without specific limitations.

After a period of time, the optical emitting module 10 stops emitting laser to the specific region SR, and the sensing module 12 is activated to perform OCT on the specific region SR after the laser treatment. Again and again, the optimal speckle removing process most suitable for the patient can be finally achieved to smoothly remove the speckle, mole, or tattoo distributed on the specific region and the tissue below it, and the patient will not feel uncomfortable; therefore, the drawbacks of the conventional laser speckle removing process can be effectively improved.

Another embodiment of the invention is an optical apparatus operating method. In this embodiment, the optical apparatus is used to emit a laser to treat a specific region of a skin surface, such as removing the speckle, mole, or tattoo distributed on the specific region and the tissue below it, but not limited to this.

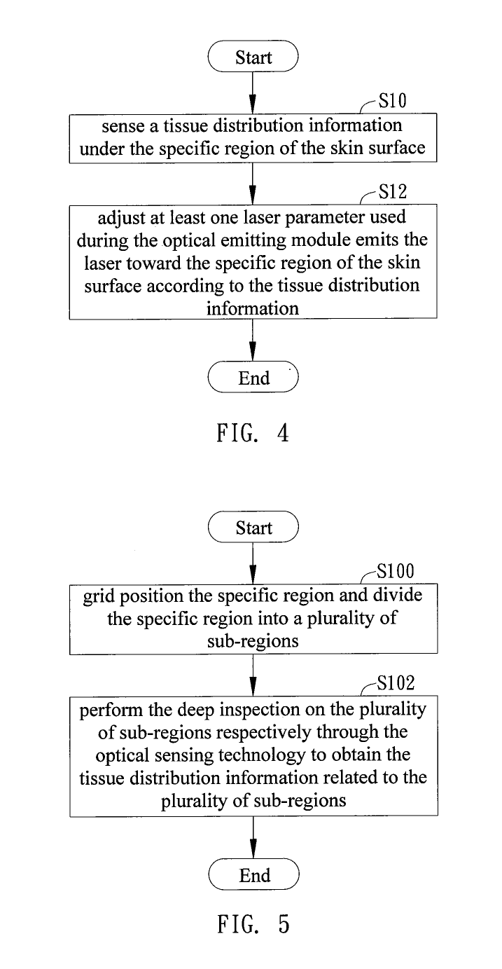

Please refer to FIG. 4. FIG. 4 illustrates a flowchart of the optical apparatus operating method in this embodiment. As shown in FIG. 4, at first, the method performs step S10 to sense a tissue distribution information under the specific region of the skin surface. Then, the method performs step S12 to adjust at least one laser parameter used during the optical emitting module emits the laser toward the specific region of the skin surface according to the tissue distribution information.

In practical applications, the at least one laser parameter includes a spot size, a wavelength, an emission energy, and an action time used during the optical emitting module emits the laser, but not limited to this.

In step S10, the method can directly perform a deep inspection on the specific region of the skin surface through an optical sensing technology or divide the specific region of the skin surface into a plurality of sub-regions at first, and then perform the deep inspection on the plurality of sub-regions respectively. In practical applications, the above-mentioned optical sensing technology can be the optical interference technology (e.g., the OCT technology), the longitudinal inspection depth is usually 2-3 mm, and the wavelength of the light used can be 1300 nm or 840 nm, but not limited to this.

For example, as shown in FIG. 5, in step S10, the method can firstly perform step S100 to grid position the specific region and divide the specific region into a plurality of sub-regions. Then, the method can perform step S102 to perform the deep inspection on the plurality of sub-regions respectively through the optical sensing technology to obtain the tissue distribution information related to the plurality of sub-regions. In fact, the method can also divide the specific region into the plurality of sub-regions in other ways; it is not limited to the grid positioning method used in this embodiment.

It should be noticed that the above-mentioned step S100 is not the necessary step of the method, that is to say, if the operator determines the exact position of the specific region through visual or based his/her experience, it is not necessary for the method to perform the grid positioning process on the specific region, and the method can directly perform the deep inspection on the plurality of sub-regions through the optical sensing technology to obtain the tissue distribution information below the specific region.

Next, the flowchart of laser speckle removing will be used to introduce the actual operation processes of the optical apparatus operating method.

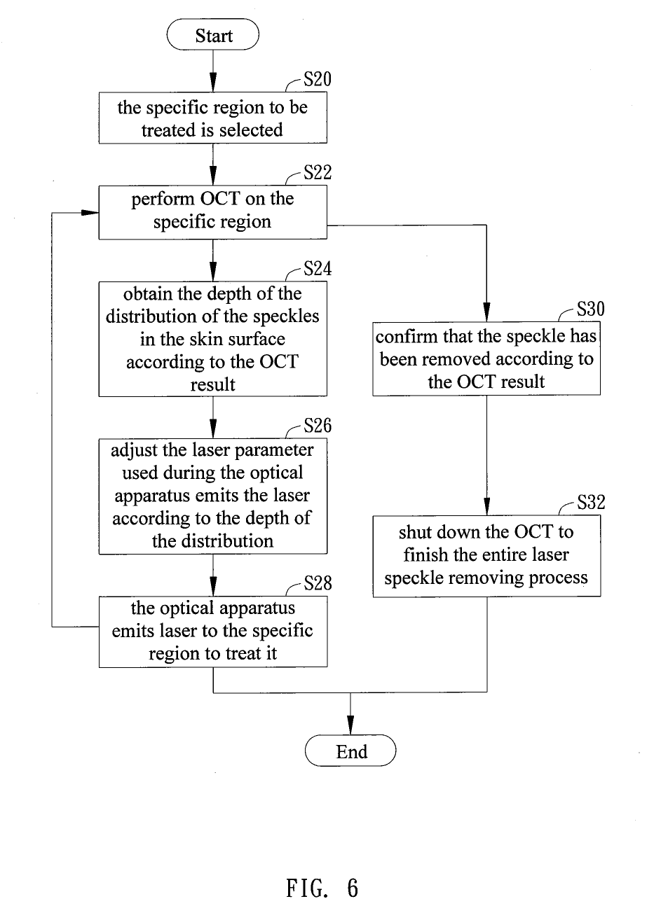

As shown in FIG. 6, after the specific region to be treated is selected (step S20), the method will perform the OCT on the specific region (step S22). Then, the method will obtain the depth of the distribution of the speckles in the skin surface according to the OCT result (step S24), and adjust the laser parameter used during the optical apparatus emits the laser according to the depth of the distribution (step S26). Afterward, the optical apparatus will emit laser to the specific region to treat it (step S28).

After a period of time, the optical apparatus will stop emitting laser, and the method will perform step S22 again to perform OCT on the specific region. If the method confirms that the speckle has been removed according to the OCT result (step S30), then the method will shut down the OCT to finish the entire laser speckle removing process (step S32). Similarly, in practical applications, the method can also treat the mole or tattoo on the skin through the above-mentioned ways to finish the process of laser removing mole or tattoo.

Compared to the prior arts, the optical apparatus and operating method thereof disclosed in the invention uses the optical interference technology (e.g., the OCT technology) to perform a deep inspection on the treated region of the skin surface, so that the actual speckle and mole distribution of the patient can be obtained, and the laser parameter used during the optical apparatus emits the laser can be adjusted accordingly to reach the optimal condition for the patient.

Therefore, the optical apparatus and operating method thereof disclosed in the invention can effectively avoid the conditions of human discomfort (excessive laser energy) or entire process delay (insufficient laser energy) caused by the errors or subjective factors of the operator, not only the drawbacks of conventional laser speckle removing process can be effectively improved, but also the satisfaction of the consumers to the laser speckle removing process can be largely increased.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. An optical apparatus, comprising:
an optical emitting module, used to emit a laser toward a specific region of a skin surface;
a sensing module, used to sense a tissue distribution information under the specific region of the skin surface; and
a processing module, coupled to the optical emitting module and the sensing module, the processing module being used to adjust at least one laser parameter used during the optical emitting module emits the laser toward the specific region of the skin surface according to the tissue distribution information.

2. The optical apparatus of claim 1, wherein the at least one laser parameter comprises a spot size, a wavelength, an emission energy, and an action time used during the optical emitting module emits the laser.

3. The optical apparatus of claim 1, wherein the sensing module performs a deep inspection on the specific region of the skin surface to obtain the tissue distribution information.

4. The optical apparatus of claim 3, wherein the sensing module performs the deep inspection on the specific region of the skin surface through an optical interference technology.

5. The optical apparatus of claim 1, wherein the sensing module divides the specific region of the skin surface into a plurality of sub-regions, and then the sensing module performs the deep inspection on the plurality of sub-regions respectively to obtain the tissue distribution information.

6. A method of operating an optical apparatus, the optical apparatus emitting a laser to treat a specific region of a skin surface, the method comprising steps of:
(a) sensing a tissue distribution information under the specific region of the skin surface; and
(b) adjusting at least one laser parameter used during the optical emitting module emits the laser toward the specific region of the skin surface according to the tissue distribution information.

7. The method of claim 6, wherein the at least one laser parameter comprises a spot size, a wavelength, an emission energy, and an action time used during the optical emitting module emits the laser.

8. The method of claim 6, wherein the step (a) comprises:
performing a deep inspection on the specific region of the skin surface through an optical sensing technology to obtain the tissue distribution information.

9. The method of claim 8, wherein the optical sensing technology is an optical interference technology.

10. The method of claim 6, wherein the step (a) comprises:
(a1) dividing the specific region of the skin surface into a plurality of sub-regions; and
(a2) performing the deep inspection on the plurality of sub-regions respectively to obtain the tissue distribution information.
